(19) United States
(12) Patent Application Publication
(10) Pub. No.: US 2025/0299791 A1
(43) Pub. Date: Sep. 25, 2025

(54) ARTIFICIAL INTELLIGENCE (AI)-DRIVEN MIXED-INITIATIVE DIALOGUE DIGITAL MEDICAL ASSISTANT

(71) Applicant: Quantum AI LLC, La Jolla, CA (US)

(72) Inventor: Dean Weber, La jolla, CA (US)

(73) Assignee: Quantum AI LLC, La Jolla, CA (US)

(21) Appl. No.: 19/083,205

(22) Filed: Mar. 18, 2025

Related U.S. Application Data
(60) Provisional application No. 63/567,295, filed on Mar. 19, 2024.

(51) Int. Cl. G16H 10/60; G10L 15/16; G10L 15/18; G10L 15/183; G10L 15/22; G10L 15/30; G10L 25/66

(52) U.S. Cl. CPC G16H 10/60; G10L 15/16; G10L 15/1822; G10L 15/183; G10L 15/22; G10L 15/30; G10L 25/66

(57) ABSTRACT

In accordance with at least one aspect of this disclosure, an artificial intelligence driven bi-directional medical assistant is provided. The assistant comprises an input module configured to recognize, in real time, spoken language and convert the spoken language to a computer readable form to generate a patient embedding. The computer readable form includes, in certain embodiments, a mathematical vector associated with the patient embedding, and the spoken language includes a conversation between a clinician and a patient during a patient visit.

### CROSS-REFERENCE TO RELATED APPLICATIONS

This application claims priority to and the benefit of U.S. Provisional Application No. 63/567,295, filed Mar. 19, 2024, the entire contents of which are herein incorporated by reference in their entirety.

### TECHNICAL FIELD

The present disclosure relates to digital assistants, and more particularly to, AI-driven mixed-initiative dialogue digital assistants, e.g., for use in the medical field.

### BACKGROUND

The practice of medicine involves numerous administrative tasks in performing and documenting patient care. The requirement of extensive documentation contributes to a reduction in medical professionals' workflow, an increase in their workload, and an increase in their burnout.

There is an ever-present need for improved systems and methods for assisting medical professionals to increase their workflow, decrease their workload, and decrease their burnout. This disclosure provides a solution for this need.

### SUMMARY

In accordance with at least one aspect of this disclosure, an artificial intelligence driven bi-directional medical assistant is provided. The assistant comprises an input module configured to recognize, in real time, spoken language and convert the spoken language to a computer readable form to generate a patient embedding. The computer readable form includes, in certain embodiments, a mathematical vector associated with the patient embedding, and the spoken language includes a conversation between a clinician and a patient during a patient visit.

The assistant further includes a memory configured to store the patient embedding in a database of existing patient embeddings and an analytics module. The analytics module is configured to, in real time use one or more artificial intelligence based analytical techniques to parse the patient embedding and catalog portions of the patient embedding into a plurality of categories including patient history, patient symptoms, patient condition, patient medication, patient allergy, patient concerns, likely medical billing codes for services rendered during the patient visit. Any number of additional desired categories may be included, and in certain embodiments, the plurality of categories may be customized by a user. In certain embodiments, the analytics modules is further configured to pass the input data to a natural language processing module, a natural language understanding module, a large language model module, a neural network module, a mixed-initiative dialogue manager module.

The analytics module is further configured to compare the patient embedding to the database of existing patient embeddings and determine a confidence matching score of the patient embedding relative to one or more existing patient embeddings. The analytics module is also configured to automatically generate a set of post visit instructions and automatically generate a patient post visit record based at least in part on one or more existing patient embeddings having a confidence matching score greater than or equal to a first predetermined threshold.

The assistant further includes an output module configured to, in real time, provide to the clinician via one or more different media the patient post visit record in a standardized format and automatically execute the set of post visit administrative instructions.

In certain embodiments, the mathematical vector includes a mathematical representation of one or more datapoints in a multidimensional space, the one or more data points including the patient history, the patient symptoms, the patient condition, the patient medication, the patient allergy, the patient concerns, and/or the likely medical billing codes for services rendered during the patient visit, among others. As discussed herein, the one or more data points can be configured for a particular application, such as a particular medical practice, or based on user configuration settings as desired for a particular user (e.g., clinician, or clinician office, or hospital). The input module is configured to convert unstructured data to structured data, wherein the patient embedding is the structured data. In certain embodiments, the structured data can include a json file.

In certain embodiments, the input module is further configured to, in real time, recognize one or more of: written language, (e.g., a patient existing record and/or clinician notes generated during the patient visit), imagery (e.g., a patient imaging existing record and/or patient images generated during the patient visit), gestures (e.g., gestures include gestures performed by a clinician during the patient visit), and/or non-spoken language acoustics (e.g., non-spoken language acoustics generated by the patient during the patient visit.

The input module can be configured to perform one or more of: speech recognition, gesture recognition, image recognition, optical character recognition, and/or acoustic recognition on the spoken language, the written language, the imagery, the gestures, and the non-spoken language acoustics. Based on the recognition, the input module is configured to convert the written language, the imagery, the gestures, and the non-spoken language acoustics to a respective mathematical vector that is associated with the generated patient embedding. The analytics module is configured to automatically update the patient embedding with the respective mathematical vectors as the input is captured, such as before the patient visit, during the patient visit, and/or after the patient visit.

In certain embodiments, the input, e.g., the spoken language, the written language, the gestures, and/or the non-spoken language acoustics is captured by the input module via an input device operatively connected (e.g., wirelessly or wired) thereto. In certain embodiments, the input device can include one or more of: a computerized medical equipment, a laptop, a desktop computer, a smart speaker, an internet browser, a mobile device, a tablet, a smart watch, smart glasses, an AR headset, a VR headset, and/or a XR headset.

In certain embodiments, the output module can be configured to perform one or more of: speech synthesis, image generation, and/or document generation to generate and provide the patient post visit record via the one or more different media. In certain embodiments, the one or more different media include can include one or more of: visual output, haptic output, and/or auditory output. The output module is configured to provide the visual output and/or auditory output to the clinician via an output device. In certain embodiments, the output device can include on one or more of: a computerized medical equipment, a laptop, a desktop computer, a smart speaker, an internet browser, a mobile device, a tablet, a smart watch, smart glasses, an AR headset, a VR headset, and/or a XR headset.

In certain embodiments, the output module can be configured to provide the clinician via the one or more different media the patient post visit record, or other information during the patient visit, in a manner that is not readily accessible to the patient during the visit. Said differently, the output module is configured to provide the clinician with information during the visit, and provide the post visit report in such a manner so that the patient does not hear or sec any display or dialogue between the clinician and the medical assistant.

The standardized format of the patient post visit record can include: a chief complaint, a subjective description, an objective description, an assessment, and a plan, for example so the standardized format is compatible with existing electronic health record databases. In certain embodiments, the chief complaint, the subjective description, and the objective description can be automatically generated from directly the patient embedding prior to the comparison to existing patient embeddings. The assessment and plan can be generated automatically by the analytics module in one or more different manners.

In certain embodiments, the assessment and the plan are automatically generated directly from the patient embedding prior to the comparison to existing patient embeddings. In certain such embodiments, the analytic module is further configured to automatically generate a secondary patient post visit record including, the chief complaint, the subjective description, the objective description, a secondary assessment, and a secondary plan. Here, the secondary assessment and the secondary plan are automatically generated by the analytic module based on the comparison of the patient embedding to the existing patient embeddings where a confidence matching score of the patient embedding to one or more existing patient embeddings is greater than or equal to a second predetermined threshold. In such embodiments, the clinician can generate the primary assessment and plan based on their observations during the patient visit, while the medical assistant provides a secondary report, or a second opinion, based on its comparison to the database of patient embeddings.

In certain embodiments, the assessment and the plan are automatically generated by the analytic module based on the comparison of the patient embedding to the existing patient embeddings where a confidence matching score of the patient embedding to one or more existing patient embeddings is greater than or equal to a second predetermined threshold. In such embodiments, the analytics module generates only the primary assessment and plan, for example if the confidence score is above a particular threshold, or the clinician agrees with the assessment and plan generated by the medical assistant.

In certain embodiments, the set of post visit administrative instructions can include one or more of inputting or uploading information from the patient post visit record to an electronic heath records database, updating an existing patient record for the patient with information from the patient post visit record, generating a referral letter to a specialty clinician, generating or beginning a pre-authorization process for follow up appointments or procedures, scheduling subsequent appointments for the patient with the clinician or with other clinicians based on information from the patient post visit record, sending a prescription order to a pharmacy based on information from the patient post visit record, coding and/or entering clinician services performed into an electronic billing system, and/or generating and providing patient friendly format of the patient post visit record to the patient before discharge. In certain embodiments, as discussed further hereinbelow, the post visit administrative instructions can be changed or defined by the information gathered during the patient visit, or may be, at least in part, standardized based on the needs of a particular clinician or particular medical practice. The list may also be, at least in part, informed by historical trends for similar patient embeddings as determined by the analytics module when comparing to the database.

In certain embodiments, the analytic module is further configured to, in real time, automatically review a patient intake database and scheduling database to determine a list of patients to be seen by the clinician. For each patient in the list, the analytics module is configured to automatically review an existing patient electronic health record for each patient to be seen, and automatically provide to the clinician, based on the review of the patient intake database and existing electronic health record for a respective patient to be seen, a pre patient visit report for each patient. The pre patient visit report can include one or more of: bibliographic information of the respective patient to be seen, a medical history of the respective patient to be seen; a proposed assessment and plan to be included in the patient post visit record; a list of follow up questions to be asked of the respective patient during the visit; and/or a list of administrative tasks to be completed post patient visit. In certain embodiments, the pre patient visit report can be provided to the clinician in the standardized format, for example, the pre patient visit report can include a draft of the post patient visit report generated based on the intake information for the respective patient.

The analytic module is further configured to, in real time, automatically modify the assessment and the plan of the pre patient visit report during the patient visit based on the patient embedding and the comparison to the database of existing patient embeddings if the confidence matching score of the patient embedding relative to one or more existing patient embeddings increases indicating a better fitting assessment and plan compared to the pre patient visit report. In this way, the post patient visit report can include a revised version of the pre patient visit report.

Similarly, in certain embodiments, the analytic module is configured to, in real time, automatically update the list of questions to be asked during the patient visit based on the patient embedding and the comparison to the database of existing patient embeddings, and further, to automatically update the list of administrative tasks to be completed post patient visit based on the patient embedding and the comparison to the database of existing patient embeddings. The set of post visit instructions can include the list of administrative tasks to be completed post visit, which may be updated by the analytics module during the visit based on the information gathered during the visit.

In accordance with at least one aspect of this disclosure, a method (e.g., a method for assisting a clinician with a patient visit and associated administrative tasks) includes, recognizing, in real time, spoken language and converting the spoken language to a computer readable form to generate a patient embedding, where the computer readable form includes a mathematical vector associated with the patient embedding, and where the spoken language includes a conversation between a clinician and a patient during a patient visit. The method further includes, storing the patient embedding in a database of existing patient embeddings and using one or more artificial intelligence based analytical techniques to parse the patient embedding and catalog portions of the patient embedding into a plurality of categories including patient history, patient symptoms, patient condition, patient medication, patient allergy, patient concerns, likely medical billing codes for services rendered during the patient visit.

The method includes, comparing the patient embedding to the database of existing patient embeddings and determining a confidence matching score of the patient embedding relative to one or more existing patient embeddings and automatically generating a set of post visit instructions and automatically generating a patient post visit record based at least in part on one or more existing patient embeddings having a confidence matching score greater than or equal to a first predetermined threshold. Further, the method incudes, providing to the clinician, via one or more different media the patient post visit record in a standardized format, and automatically executing the set of post visit administrative instructions.

The method also includes, in real time, recognizing and analyzing written language, (e.g., a patient existing record and/or clinician notes generated during the patient visit), recognizing and analyzing imagery (e.g., a patient imaging existing record and/or patient images generated during the patient visit), recognizing and analyzing gestures (e.g., gestures performed by a clinician during the patient visit), and/or recognizing and analyzing non-spoken language acoustics (e.g., non-spoken language acoustics generated by the patient during the patient visit). The method further comprises converting the written language, the imagery, the gestures, and the non-spoken language acoustics to a respective mathematical vector that is associated with the generated patient embedding, and automatically updating the patient embedding with the respective mathematical vectors as the written language, the imagery, the gestures, and the non-spoken language acoustics are captured.

In certain embodiments, the standardized format of the patient post visit record includes: a chief complaint, a subjective description, an objective description, an assessment, and a plan, In certain such embodiments, the method further includes automatically generating the chief complaint, the subjective description, and the objective description directly from the patient embedding prior to the comparison to existing patient embeddings. The method also includes one or more of the following:
i) automatically generating the assessment and the plan directly from the patient embedding prior to the comparison to existing patient embeddings, and further comprising automatically generating a secondary patient post visit record including, the chief complaint, the subjective description, the objective description, a secondary assessment, and a secondary plan, where the secondary assessment and the secondary plan are automatically generated based on the comparison of the patient embedding to the existing patient embeddings where a confidence matching score of the patient embedding to one or more existing patient embeddings is greater than or equal to a second predetermined threshold, or
ii) automatically generating the assessment and the plan based on the comparison of the patient embedding to the existing patient embeddings where a confidence matching score of the patient embedding to one or more existing patient embeddings is greater than or equal to a second predetermined threshold.

The method also includes, in real time, automatically reviewing a patient intake database and scheduling database and generating a list of patients to be seen by the clinician, automatically reviewing an existing patient electronic health record for each patient to be seen, and automatically providing to the clinician, in the standardized format, based on the review of the patient intake database and existing electronic health record for a respective patient to be seen, a pre patient visit report for each patient. In certain embodiments, the pre patient visit report can include one or more of: bibliographic information of the respective patient to be seen, a medical history of the respective patient to be seen, a proposed assessment and plan to be included in the patient post visit record, a list of follow up questions to be asked of the respective patient during the visit. and/or a list of administrative tasks to be completed post patient visit.

In certain embodiments, the method further includes, in real time, automatically modifying the assessment and the plan of the pre patient visit report during the patient visit based on the patient embedding and the comparison to the database of existing patient embeddings if the confidence matching score of the patient embedding relative to one or more existing patient embeddings increases indicating a better fitting assessment and plan compared to the pre patient visit report.

These and other features of the systems and methods of the subject disclosure will become more readily apparent to those skilled in the art from the following detailed description taken in conjunction with the drawings.

### BRIEF DESCRIPTION OF THE DRAWINGS

So that those skilled in the art to which the subject disclosure appertains will readily understand how to make and use the devices and methods of the subject disclosure without undue experimentation, other embodiments thereof will be described in detail herein below with reference to certain figures, wherein:

FIG. 1 illustrates an example communication network utilized with one or more of the illustrated embodiments;

FIG. 2 illustrates an example network device/node utilized with one or more of the illustrated embodiments;

FIG. 3 illustrates a diagram depicting an Artificial Intelligence (AI) device utilized with one or more of the illustrated embodiments;

FIG. 4 illustrates a diagram depicting an AI server utilized with one or more of the illustrated embodiments;

FIG. 5 illustrates diagram depicting an exemplary embodiment of the digital medical assistant in accordance with this disclosure, showing an interaction of the medical assistant with a clinician during a patient visit;

FIG. 6 shows an exemplary process for a pre-visit service of the digital medical assistant in accordance with this disclosure;

FIG. 7 shows an exemplary process for a in-visit service of the digital medical assistant in accordance with this disclosure;

FIG. 8 shows an exemplary embodiment of an input device used by the digital medical assistant during an in-visit service in accordance with this disclosure to capture a dialogue between the clinician and the patient;

FIG. 9 shows an exemplary conversation or dialogue between the digital medical assistant and the clinician;

FIG. 10 shows a flow diagram of an exemplary embodiment of a method in accordance with this disclosure;

FIG. 11 shows an example of a patient encounter template utilized by the digital medical assistant in performing the pre-visit and/in visit service;

FIG. 12 shows an example of the digital medical assistant generating a documentation based on description of the medical exam performed by the clinician; and

FIGS. 13 and 14 show an example of elements used to generate a history of present illness to be used by the digital medical assistant when generating a patient embedding, where FIG. 13 shows exemplary fields of a history of present illness, and FIG. 14 shows exemplary tags to be used when injecting information into the history of present illness.

### DETAILED DESCRIPTION

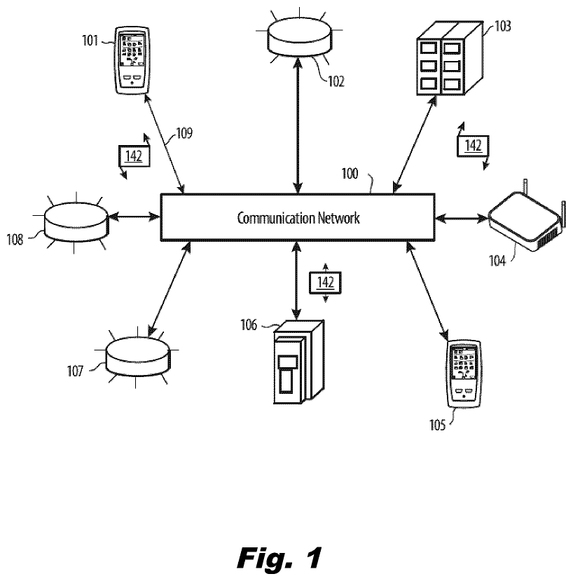

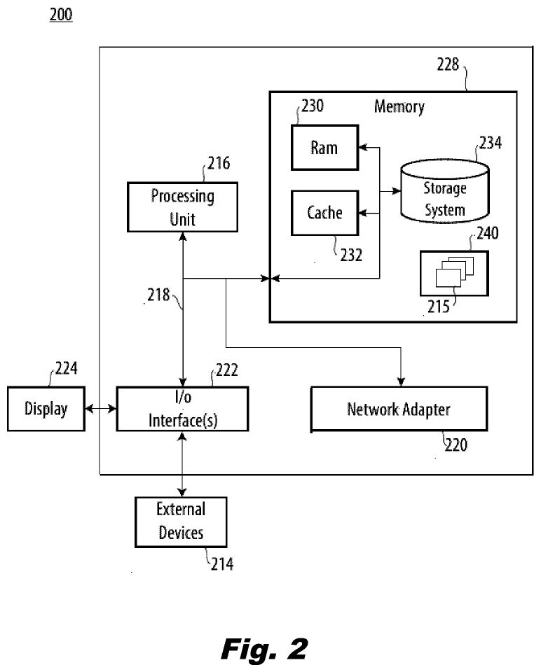

Reference will now be made to the drawings wherein like reference numerals identify similar structural features or aspects of the subject disclosure. For purposes of explanation and illustration, and not limitation, an illustrative view of an embodiment of a system in accordance with the disclosure is shown in FIG. 1 and is designated generally by reference character 100. Other embodiments and/or aspects of this disclosure are shown in FIGS. 2-14. Certain embodiments of the digital medical assistant described herein can be used to improve efficiency and accuracy of medial documentation as well as diagnosis and treatment of patients. Embodiments of the digital medical assistant provide a specific technological improvement over existing electronic medical diagnosis systems and/or processes by increasing speed, efficiency and accuracy of existing systems by and allowing the digital medical assistant described herein to access a “global” database of patient information for comparison in real time. This allows the digital medical assistant described herein to generate more complete and accurate assessment and plans for a patient during a patient visit. Further, the medical assistant described herein removes a significant portion of the clinician's administrative burden, making appointments, charting, billing, and result generation much quicker and more efficient for all parties involved. This allows the clinician to spend more quality time with the patient.

The illustrated embodiments are now described more fully with reference to the accompanying drawings wherein like reference numerals identify similar structural/functional features. The illustrated embodiments are not limited in any way to what is illustrated as the illustrated embodiments described below are merely exemplary, which can be embodied in various forms, as appreciated by one skilled in the art. Therefore, it is to be understood that any structural and functional details disclosed herein are not to be interpreted as limiting, but merely as a basis for the claims and as a representation for teaching one skilled in the art to variously employ the discussed embodiments. Furthermore, the terms and phrases used herein are not intended to be limiting but rather to provide an understandable description of the illustrated embodiments.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the illustrated embodiments, exemplary methods and materials are now described.

It must be noted that as used herein and in the appended claims, the singular forms “a”, “an,” and “the” include plural referents unless the context clearly dictates otherwise. Thus, for example, reference to “a stimulus” includes a plurality of such stimuli and reference to “the signal” includes reference to one or more signals and equivalents thereof known to those skilled in the art, and so forth.

It is to be appreciated the illustrated embodiments discussed below are preferably a software algorithm, program or code residing on computer useable medium having control logic for enabling execution on a machine having a computer processor. In accordance with the illustrated embodiments, machine learning techniques are preferably utilized for assisting in the assessment and diagnosis of a medical patient and generation of a treatment plan therefor, for example by automatically updating and comparing a patient embedding to a database of patient embeddings during a patient exam to automatically generate a proposed diagnosis and treatment plan, among other things.

As used herein, the term “software” is meant to be synonymous with any code or program that can be in a processor of a host computer, regardless of whether the implementation is in hardware, firmware or as a software computer product available on a disc, a memory storage device, or for download from a remote machine. The embodiments described herein include such software to implement the equations, relationships and algorithms described above. One skilled in the art will appreciate further features and advantages of the illustrated embodiments based on the above-described embodiments. Accordingly, the illustrated embodiments are not to be limited by what has been particularly shown and described, except as indicated by the appended claims.

Turning now descriptively to the drawings, in which similar reference characters denote similar elements throughout the several views, FIG. 1 depicts an exemplary communications network 100 in which below illustrated embodiments may be implemented. It is to be understood a communication network 100 is a geographically distributed collection of nodes interconnected by communication links and segments for transporting data between end nodes, such as personal computers, work stations, smart phone devices, tablets, televisions, sensors and or other devices such as automobiles, etc. Many types of networks are available, with the types ranging from local area networks (LANs) to wide area networks (WANs). LANs typically connect the nodes over dedicated private communications links located in the same general physical location, such as a building or campus. WANs, on the other hand, typically connect geographically dispersed nodes over long-distance communications links, such as common carrier telephone lines, optical lightpaths, synchronous optical networks (SONET), synchronous digital hierarchy (SDH) links, or Powerline Communications (PLC), and others.

FIG. 1 is a schematic block diagram of an example communication network 100 illustratively comprising nodes/devices 101-108 (e.g., sensors 102, client computing devices 103 (e.g., network monitoring devices), smart phone devices 105, web servers 106, routers 107, switches 108, databases, and the like) interconnected by various methods of communication. For instance, the links 109 may be wired links or may comprise a wireless communication medium, where certain nodes are in communication with other nodes, e.g., based on distance, signal strength, current operational status, location, etc. Moreover, each of the devices can communicate data packets (or frames) 142 with other devices using predefined network communication protocols as will be appreciated by those skilled in the art, such as various wired protocols and wireless protocols etc., where appropriate. In this context, a protocol consists of a set of rules defining how the nodes interact with each other. Those skilled in the art will understand that any number of nodes, devices, links, etc. may be used in the computer network, and that the view shown herein is for simplicity. Also, while the embodiments are shown herein with reference to a general network cloud, the description herein is not so limited, and may be applied to networks that are hardwired.

As will be appreciated by one skilled in the art, aspects of the illustrated embodiments may be embodied as a system, method or computer program product. Accordingly, aspects of the illustrated embodiments may take the form of an entirely hardware embodiment, an entirely software embodiment (including firmware, resident software, micro-code, etc.) or an embodiment combining software and hardware aspects that may all generally be referred to herein as a “device”, “apparatus”, “module” or “system.” Furthermore, aspects of the illustrated embodiments may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++, Python, or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the illustrated embodiments are described below with reference to flowchart illustrations and/or block diagrams of methods, apparatus (systems) and computer program products according to the illustrated embodiments. It will be understood that each block of the flowchart illustrations and/or block diagrams, and combinations of blocks in the flowchart illustrations and/or block diagrams, can be implemented by computer program instructions. These computer program instructions may be provided to a processor of a computer device, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

FIG. 2 is a schematic block diagram of an example network computing device 200 (e.g., client computing device 103, server 106, etc.) that may be used (or components thereof) with one or more embodiments described herein (e.g., as one of the nodes shown in the network 100) for determining the probability of an incident occurring to one or more computer applications resulting from one or more application change attributes through implementation of machine learning (ML) techniques. As explained above, in different embodiments these various devices are configured to communicate with each other in any suitable way, such as, for example, via communication network 100.

Device 200 is intended to represent any type of computer system capable of carrying out the teachings of various illustrated embodiments. Device 200 is only one example of a suitable system and is not intended to suggest any limitation as to the scope of use or functionality of the illustrated embodiments described herein. Regardless, computing device 200 is capable of being implemented and/or performing any of the functionality set forth herein, particularly for creating patient embeddings based on one or more forms of input data collected before or during a patient visit with a clinician, and comparing those embeddings to a database to quickly and accurately assist the clinician with determining an assessment and plan for the patient after the visit, and automatically executing, post visit administrative tasks, with or without further input from the clinician in accordance with the illustrated embodiments.

It is to be understood and appreciated that computing device 200 is operational with numerous other general purpose or special purpose computing system environments or configurations. Examples of well-known computing systems, environments, and/or configurations that may be suitable for use with computing device 200 include, but are not limited to, personal computer systems, server computer systems, thin clients, thick clients, hand-held or laptop devices, multiprocessor systems, microprocessor-based systems, network PCs, minicomputer systems, and distributed data processing environments that include any of the above systems or devices, and the like. Computing device 200 may be described in the general context of computer system-executable instructions, such as program modules, being executed by a computer system. Generally, program modules may include routines, programs, objects, components, logic, data structures, and so on that perform particular tasks or implement particular abstract data types. Computing device 200 may be practiced in distributed data processing environments where tasks are performed by remote processing devices that are linked through a communications network 100. In a distributed data processing environment, program modules may be located in both local and remote computer system storage media including memory storage devices.

The components of device 200 may include, but are not limited to, one or more processors or processing units 216, a system memory 228, and a bus 218 that couples various system components including system memory 228 to processor 216. Bus 218 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus. Computing device 200 typically includes a variety of computer system readable media. Such media may be any available media that is accessible by device 200, and it includes both volatile and non-volatile media, removable and non-removable media.

System memory 228 can include computer system readable media in the form of volatile memory, such as random-access memory (RAM) 230 and/or cache memory 232. Computing device 200 may further include other removable/non-removable, volatile/non-volatile computer system storage media. By way of example only, storage system 234 can be provided for reading from and writing to a non-removable, non-volatile magnetic media (not shown and typically called a “hard drive”). Although not shown, a magnetic disk drive for reading from and writing to a removable, non-volatile magnetic disk, and an optical disk drive for reading from or writing to a removable, non-volatile optical disk such as a CD-ROM, DVD-ROM or other optical media can be provided. In such instances, each can be connected to bus 218 by one or more data media interfaces. As will be further depicted and described below, memory 228 may include at least one program product having a set (e.g., at least one) of program modules that are configured to carry out the functions of embodiments of illustrated embodiments such as automatically creating/updating patient embeddings during a conversation between a patient and clinician, and automatically generating an assessment and plan in view thereof in accordance with the illustrated embodiments.

Program/utility 240, having a set (at least one) of program modules 215, such as underwriting module, may be stored in memory 228 by way of example, and not limitation, as well as an operating system, one or more application programs, other program modules, and program data. Each of the operating system, one or more application programs, other program modules, and program data or some combination thereof, may include an implementation of a networking environment. Program modules 215 generally carry out the functions and/or methodologies of the illustrated embodiments as described herein automatically creating/updating patient embeddings during a conversation between a patient and clinician, and automatically generating an assessment and plan in view thereof for output by one or more networked computer devices (e.g., 103, 106, 1110).

Device 200 may also communicate with one or more external devices 214 such as a keyboard, a pointing device, a display 224, etc.; one or more devices that enable a user to interact with computing device 200; and/or any devices (e.g., network card, modem, etc.) that enable computing device 200 to communicate with one or more other computing devices. Such communication can occur via Input/Output (I/O) interfaces 222. Still yet, device 200 can communicate with one or more networks such as a local area network (LAN), a general wide area network (WAN), and/or a public network (e.g., the Internet) via network adapter 220. As depicted, network adapter 220 communicates with the other components of computing device 200 via bus 218. It should be understood that although not shown, other hardware and/or software components could be used in conjunction with device 200. Examples, include, but are not limited to: microcode, device drivers, redundant processing units, external disk drive arrays, RAID systems, tape drives, and data archival storage systems, etc.

FIGS. 1 and 2 are intended to provide a brief, general description of an illustrative and/or suitable exemplary environment in which the below described illustrated embodiments may be implemented. FIGS. 1 and 2 are exemplary of a suitable environment and are not intended to suggest any limitation as to the structure, scope of use, or functionality of an illustrated embodiment. A particular environment should not be interpreted as having any dependency or requirement relating to any one or combination of components illustrated in an exemplary operating environment. For example, in certain instances, one or more elements of an environment may be deemed not necessary and omitted. In other instances, one or more other elements may be deemed necessary and added.

It is to be understood the embodiments described herein are preferably provided with Machine Learning (ML)/Artificial Intelligence (AI) techniques for automatically creating/updating patient embeddings during a conversation between a patient and clinician, and automatically generating an assessment and plan in view thereof in accordance with the illustrated embodiments through implementation of AI techniques. The computer system 200 is preferably integrated with an AI system (as also described below) that is preferably coupled to a plurality of external databases/data sources that implements machine learning and artificial intelligence algorithms in accordance with the illustrated embodiments. For instance, the AI system may include two subsystems: a first sub-system that learns from historical data; and a second subsystem to identify and recommend one or more parameters or approaches based on the learning for detecting anomaly events in computer devices. It should be appreciated that although the AI system may be described as two distinct subsystems, the AI system can also be implemented as a single system incorporating the functions and features described with respect to both subsystems.

Also in accordance with certain illustrated embodiments, a neural network (NN) may be used as the trained ML model for more accurate suggestions with respect to diagnosis a patient based on the information gathered during the pre-and during-visit conversations between the patient and clinician in accordance with the illustrated embodiments. It is to be appreciated that a neural network is a model used in machine learning and may mean a whole model of problem-solving ability which is composed of artificial neurons (nodes) that form a network by synaptic connections. The artificial neural network can be defined by a connection pattern between neurons in different layers, a learning process for updating model parameters, and an activation function for generating an output value. The artificial neural network preferably includes an input layer, an output layer, and one or more hidden layers. Each layer includes one or more neurons, and the artificial neural network may include a synapse that links neurons to neurons. In the artificial neural network, each neuron may output the function value of the activation function for input signals, weights, and deflections input through the synapse.

It is to be understood and appreciated that model parameters refer to parameters determined through learning and include a weight value of synaptic connection and deflection of neurons. A hyperparameter means a parameter to be set in the machine learning algorithm before learning, and typically includes a learning rate, a repetition number, a mini batch size, and an initialization function. The purpose of the learning of the neural network may be to determine the model parameters that minimize a loss function. The loss function may be used as an index to determine optimal model parameters in the learning process of the neural network. Machine learning may be classified into supervised learning, unsupervised learning, and reinforcement learning according to a learning method. The supervised learning may refer to a method of learning a neural network in a state in which a label for learning data is given, and the label may mean the correct answer (or result value) that the neural network must infer when the learning data is input to the artificial neural network. The unsupervised learning may refer to a method of learning a neural network in a state in which a label for learning data is not given. The reinforcement learning may refer to a learning method in which an agent defined in a certain environment learns to select a behavior or a behavior sequence that maximizes cumulative compensation in each state.

Is it to also be appreciated that machine learning, which is implemented as a deep neural network (DNN) including a plurality of hidden layers among neural networks, is also referred to as deep learning, and the deep learning is part of machine learning.

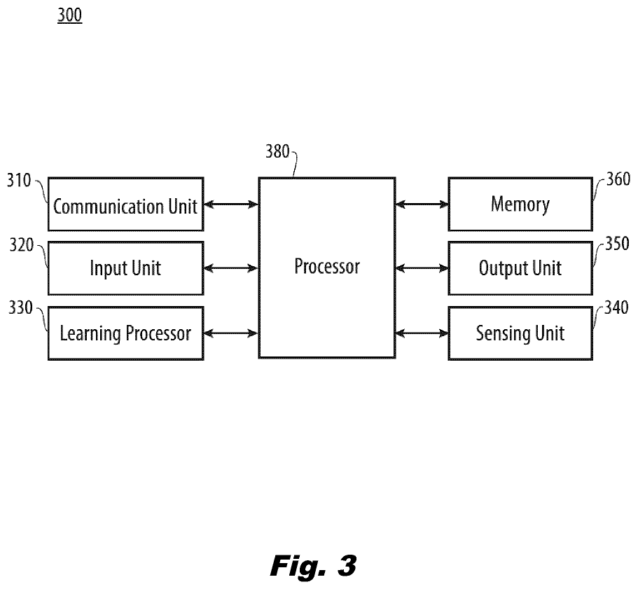

Referring now to FIG. 3, it illustrates an Artificial Intelligence (AI) monitoring device 300 according to an embodiment of the illustrated embodiments. The AI monitoring device 300 may be implemented by a stationary device or a mobile device, such as a web server, a desktop computer, a notebook, a desktop computer, and the like.

In conjunction with FIGS. 1 and 2, the AI monitoring device 300 of FIG. 3 is operatively coupled to, or integrated with computing device 200, in accordance with the illustrated embodiments described herein. AI monitoring device 300 preferably includes a communication unit 310, an input unit 320, a learning processor 330, a sensing unit 340, an output unit 350, a memory 360, and a processor 380. The communication unit 310 may transmit and receive data to and from external devices, such as other AI devices, by using wire/wireless communication technology. For example, the communication unit 310 may transmit and receive historical and contemplated application change attributes, a user input, a learning model, and a control signal to and from external devices, such as AI server 400.

The communication technology used by the communication unit 310 preferably includes GSM (Global System for Mobile communication), CDMA (Code Division Multi Access), LTE (Long Term Evolution), 5G, WLAN (Wireless LAN), Wi-Fi (Wireless-Fidelity), Bluetooth™, RFID (Radio Frequency Identification), Infrared Data Association (IrDA), ZigBee, NFC (Near Field Communication), and the like.

In accordance with the illustrated embodiments, the input unit 320 may acquire various kinds of data, including, but not limited to patient health records, electronic health records, dialogue between the clinician and the patient, lab work, imaging studies, or the like, to be used to generate a patient embedding and, ultimately, a patient post visit record, including an assessment and plan based on a diagnosis, generated at least partially by the medical assistant 1120. The input unit 320 may acquire a learning data for model learning (e.g., existing patient embeddings stored for future model training to improve diagnosis accuracy) and input data to be used when an output is acquired by using a learning model. The input unit 320 may acquire raw input data. The ML model in certain embodiments infers a result value for new input data rather than learning data, and the inferred value may be used as a basis for determination to perform a certain operation.

In certain illustrated embodiments, the learning processor 330 performs AI processing together with the learning processor 440 of the AI server 400, and the learning processor 330 may include a memory integrated or implemented in the AI monitoring device 300. Alternatively, in other illustrated embodiments, the learning processor 330 is implemented by using the memory 360, an external memory directly connected to the AI monitoring device 300, or a memory held in an external device.

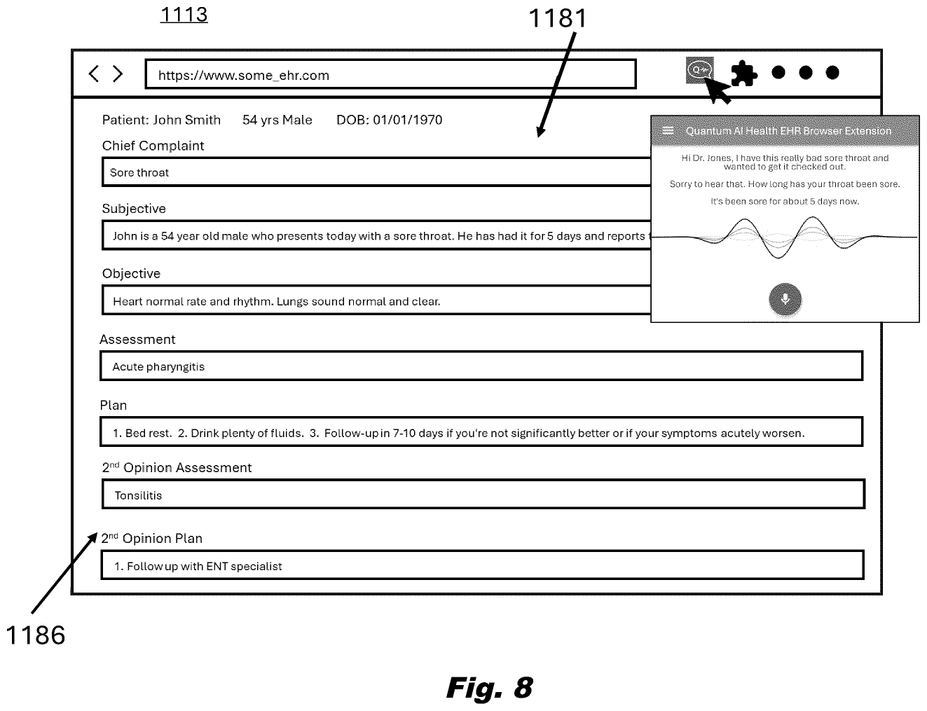

The output unit 350 preferably includes a display unit for outputting/displaying relevant information to a user in accordance with the illustrated embodiments described herein (e.g., the exemplary dashboard shown in FIG. 8 for example). The memory 360 preferably stores data that supports various functions of the AI monitoring device 300. For example, the memory 360 may store input data acquired by the input unit 320, learning data, a learning model, a learning history, and the like.

The processor 380 preferably determines at least one executable operation of the AI monitoring device 300 based on information determined or generated by using a data analysis algorithm or a machine learning algorithm. The processor 380 may control the components of the AI monitoring device 300 to execute the determined operation. To this end, the processor 380 may request, search, receive, or utilize time-based metric data of the learning processor 330 or the memory 360. The processor 380 may control the components of the AI monitoring device 300 to execute the predicted operation or the operation determined to be desirable among the at least one executable operation. When the connection of an external device is required to perform a determined operation, the processor 380 may generate a control signal for controlling the external device and may transmit the generated control signal to the external device. The processor 380 may acquire intention information for the user input and may determine the user's requirements based on the acquired intention information. In some embodiments, the processor 380 may acquire the intention information corresponding to the user input by using at least one of a speech to text (STT) engine for converting speech input into a text string or a natural language processing (NLP) engine for acquiring intention information of a natural language.

In certain illustrated embodiments, at least one of the STT engine or the NLP engine may be configured as an artificial neural network, at least part of which is learned according to the machine learning algorithm. Thus, in certain illustrated embodiments, at least one of the STT engine or the NLP engine may be learned by the learning processor 330 or may be learned by the learning processor 340 of the AI server 400, or may be learned by their distributed processing. The processor 380 may collect history information including the operation contents of the AI monitoring device 300 or the user's feedback on the operation and may store the collected history information in the memory 360 or the learning processor 330 or transmit the collected history information to the external device such as the AI server 400. The collected history information may be used to update the learning model.

The processor 380 may control at least part of the components of AI monitoring device 300 so as to drive an application program stored in memory 360. Furthermore, the processor 380 may operate two or more of the components included in the AI monitoring device 300 in combination so as to drive the application program.

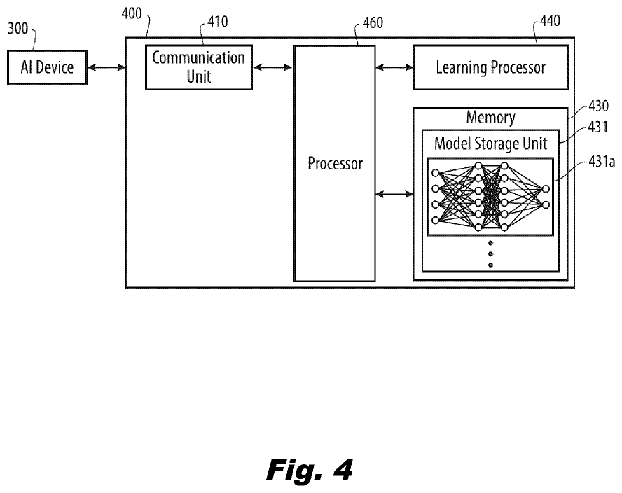

FIG. 4 illustrates an AI server 400 according to the certain illustrated embodiments that may utilize a neural network for ML. It is to be appreciated that the AI server 400 may refer to a device that learns an artificial neural network by using a machine learning algorithm or uses a learned artificial neural network. The AI server 400 may include a plurality of servers to perform distributed processing, or may be defined as a 5G network. Preferably, the AI server 400 is included as a partial configuration of the AI monitoring device 300, and performs at least part of the AI processing together. The Al server 400 may include a communication unit 410, a memory 430, a learning processor 440, a processor 460, and the like. The communication unit 410 can transmit and receive data to and from an external device such as the AI monitoring device 300. The memory 430 may include a model storage unit 431. The model storage unit 431 may store a learning or learned model (or a neural network 431a) through the learning processor 440.

The learning processor 440 may learn the artificial neural network 431a by using the learning data. The learning model may be used in a state of being mounted on the AI server 400 of the neural network or may be used in a state of being mounted on an external device such as the AI monitoring device 300. The learning model may be implemented in hardware, software, or a combination of hardware and software. If all or part of the learning models are implemented in software, one or more instructions that constitute the learning model may be stored in memory 430. The processor 460 may infer the result value for new input data by using the learning model and may generate a response or a control command based on the inferred result value.

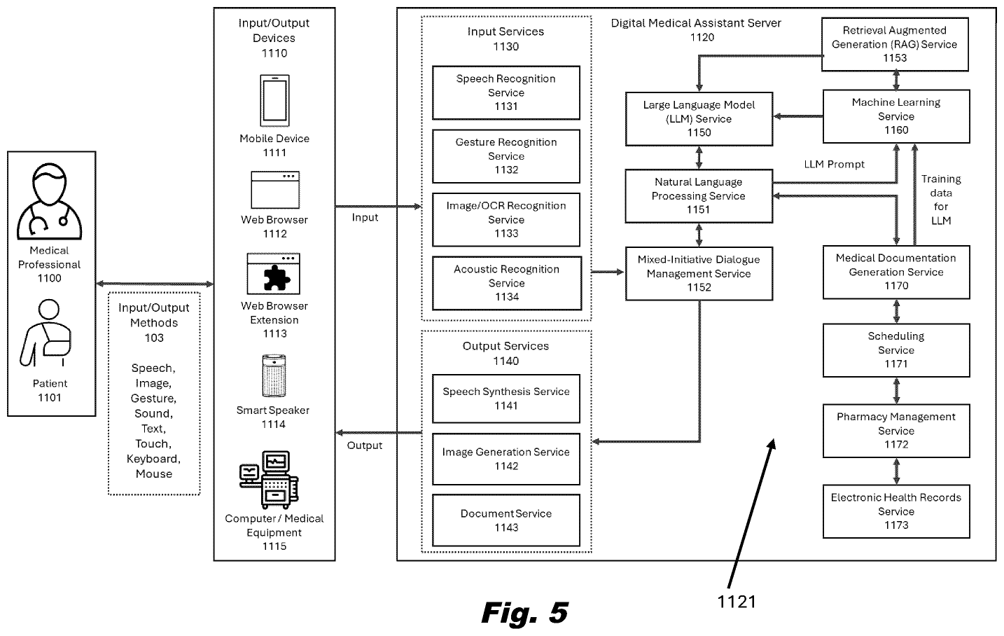

In accordance with at least one aspect of this disclosure, as shown in FIG. 5, an artificial intelligence driven bi-directional medical assistant 1120 is provided. The assistant 1120 comprises an input module 1130 configured to recognize, in real time, spoken language (e.g., a conversation between a clinician 1100 and a patient 1101 during a patient visit), written language, (e.g., a patient existing record and/or clinician notes generated during the patient visit), imagery (e.g., existing patient images and/or patient images generated during the patient visit including, pictures, x-rays or scans, ultrasounds, ECGs and the like), gestures (e.g., gestures performed by a clinician during the patient visit such as eye tracking and motion tracking, or gestures performed by the patient including involuntary movements, reflexes, range of motion or the like), and non-spoken language acoustics (e.g., non-spoken language acoustics generated by the patient during the patient visit such as breath sounds, heat beat, or sounds generated by the exam such as percussive sounds or the like). The spoken language, written language, imagery, gestures, and non-spoken language acoustics will be hereinafter referred to generally as “input data.” The input module 1130 is configured to receive the input data through an input device 1110 and convert the input to a computer readable form to generate a patient embedding for the respective patient.

As used herein, “clinician” includes any medical professional having interaction with a patient, including a doctor, a physician, a nurse, a laboratory or testing technician, a physician's assistant, or the like. As used herein a “visit” or “appointment” can include any medical exam including in person or virtually performed via telehealth appointment or can include a medical procedure where both clinician and patient are together, such as a surgical procedure, imaging study, or other testing.

In certain embodiments, the computer readable form includes a mathematical vector associated with the patient embedding. The mathematical vector includes a mathematical representation of one or more datapoints in a multidimensional space, the one or more data points including the patient history, the patient symptoms, the patient condition, the patient medication, the patient allergy, the patient concerns, and/or the likely medical billing codes for services rendered during the patient visit, among others. As discussed herein, the one or more data points can be configured for a particular application, such as a particular medical practice, or based on user configuration settings as desired for a particular user (e.g., clinician, or clinician office, or hospital). The input module 1130 is configured to convert unstructured data to structured data, wherein the patient embedding is the structured data. In certain embodiments, the structured data can include a json file.

The input module 1130 can be configured to perform one or more of: speech recognition, gesture recognition, image recognition, optical character recognition, and/or acoustic recognition on the input data and automatically update the patient embedding with the respective mathematical vectors as the input data is captured in real time, such as before the patient visit, during the patient visit, and/or after the patient visit, as will be discussed further below.

In certain embodiments, input device 1110 operatively connected (e.g., wirelessly or wired) to the input module 1130 can include one or more of: a laptop 1111, a desktop computer, a smart speaker 1114, an internet browser 1113, a mobile device 1111, a tablet 1111, a smart watch 1111, smart glasses 1111, an AR headset 1111, a VR headset 1111, a XR headset 1111, and/or a computerized medical equipment 1115.

The medical assistant 1120 further includes a memory i.e., an embedding database 1184 configured to store the patient embeddings. The database 1184 includes a historical records of existing patient embeddings. The medical assistant 1120 is configured to, in real time, use one or more artificial intelligence based analytical techniques to parse the patient embedding and catalog portions of the patient embedding into a plurality of categories. The plurality of categories can include patient bibliographic and personal information, patient history, patient symptoms (e.g., chief complaint), patient condition, patient medication, patient allergy, patient concerns, likely medical billing codes for services rendered during the patient visit, or the like. Any number of additional desired categories may be included, and in certain embodiments, the plurality of categories may be customized by a user. In certain embodiments, the analytics modules 1121 is further configured to pass the input data to a natural language processing module, a natural language understanding module, a large language model module, a neural network module, a mixed-initiative dialogue manager module when performing the one or more AI based analytical techniques.

Before, during, or after the patient visit, the analytics module 1121 is further configured to compare the patient embedding to the database 1184 of existing patient embeddings and determine a confidence matching score of the patient embedding relative to one or more existing patient embeddings. The analytics module 1121 is also configured to automatically generate a set of post visit instructions and automatically generate a patient post visit record based at least in part on one or more existing patient embeddings having a confidence matching score greater than or equal to a first predetermined threshold. For example, the analytics module 1121 can compare the patient embedding generated at any point during a patient visit (including during pre-visit review, live during the visit, or after the visit is complete) to all existing patient embeddings in the database 1184. This comparison can inform the data included on the patient post visit record and the post visits instructions.

A confidence threshold, or sensitivity, can be set by a user so that the analytics module 1121 compares to patient embeddings having a similar or exact set of characteristics, such as age, sex, family history, and medication history. This can improve the efficiency of the system. Then, when comparing the patient embedding of the current patient to the database 1184, or the subset defined by the sensitivity, the analytics module 1121 can determine how many data points match, and set the confidence score based thereon, wherein a high confidence matching score indicates many matching datapoints. The analytics module 1121 can then determine a proposed diagnosis, prognosis, outcome, or the like, for the current patient, based the patient embedding of the highly matched existing patient embedding(s). Therefore, a high confidence matching score can correlate to a high likelihood of accurate diagnosis of the patient. This will be discussed further later.

The medical assistant 1120 further includes an output module 1140 configured to, in real time, provide to the clinician the patient post visit record in a standardized format. In certain embodiments, the output module 1140 can be configured to perform one or more of: speech synthesis, image generation, and/or document generation to generate and provide the patient post visit record via one or more different media formats. In certain embodiments, the one or more different media include can include one or more of: printed material, visual output, haptic output, and/or auditory output.

The output module 1140 is configured to provide the visual output and/or auditory output to the clinician via an output device which may be one or more of the devices 1110. In certain embodiments, the output device 1110 can include on one or more of: a computerized medical equipment, a laptop, a desktop computer, a smart speaker, an internet browser, a mobile device, a tablet, a smart watch, smart glasses, an AR headset, a VR headset, and/or a XR headset.

The output module 1140 is also configured to, in real time, automatically execute the set of post visit administrative instructions, for example once the visit ends, or otherwise once the instructions are able to be completed. For example, certain tasks may be completed during the visit, such as preparing or calling in medications, scheduling follow up visits with specialists, or preparing insurance pre-authorizations may occur in real time as the clinician instructs the assistant. Other tasks, like preparing a discharge report can be partially completed in real time as information becomes available but will not be provided to the patient until the visit is complete.

In certain embodiments, the output module 1140 can be configured to provide the clinician via the one or more different media the patient post visit record, or other information during the patient visit, in a manner that is not readily accessible to the patient during the visit. Said differently, the output module 1140 is configured to provide the clinician with information during the visit and provide the post visit report in such a manner so that the patient does not hear or see any display or dialogue between the clinician 1100 and the medical assistant 1120. This can be particularly useful or advantageous when the medical assistant 1120 generates certain sensitive information, such as a terminal diagnosis, where the assessment should come from the clinician directly.

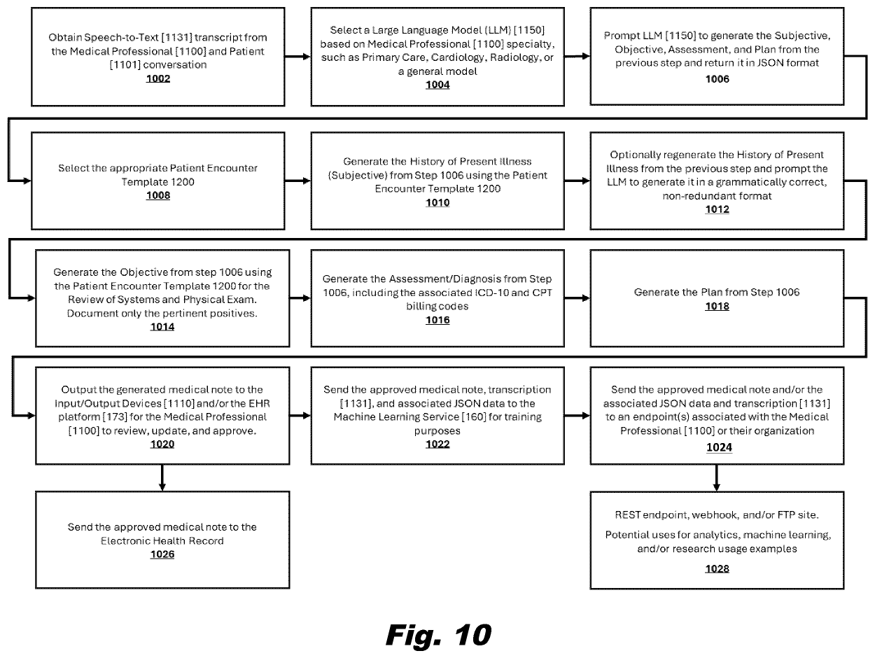

The standardized format of the patient post visit record generated by the output module 1140 can include: a chief complaint, a subjective description, an objective description, an assessment, and a plan, for example so the standardized format is easily compatible with existing electronic health record databases. In certain embodiments, the chief complaint, the subjective description, and the objective description can be automatically generated from directly the patient embedding prior to the comparison to existing patient embeddings. Since the chief complaint (i.e. the patients' symptoms as described to the clinician), the subjective description (i.e., the patient's medical history, information regarding the onset of the chief complaint, or other symptoms), and the object description (e.g., lab work, blood tests, imaging studies, or information discovered during the exam) are relevant only to the patient, there is no need to compare this input data to the database 1184 of patient embeddings. Therefore, the medical assistant 1120 can capture this input data using one of the methods described herein and automatically enter the input data into the relevant fields of the patient post visit record, without performing any analytics. This is discussed further with reference to FIGS. 10-14.

Since the assessment and plan are derived from the information gathered during the visit, and are informed by the chief complaint, the subjective description, and the objective description, it is advantageous to have the analytics module 1121 automatically generate the assessment and plan to be included in the patient post visit record. The assessment and plan can be generated automatically by the analytics module 1121 in one or more different manners.

In certain embodiments, the assessment and the plan are automatically generated directly from the patient embedding prior to the comparison to existing patient embeddings but may be manually modified by the clinician based on their personal expertise or consult and in view of their understanding of the input data gathered before and during the patient visit. Here, the analytics module 1121 can automatically generate a secondary assessment and secondary plan based on the comparison of the patient embedding to the existing patient embeddings where a confidence matching score of the patient embedding to one or more existing patient embeddings is greater than or equal to a second predetermined threshold (for example, in certain embodiments, where the confidence matching score is greater than 75%, 85% or more). In such embodiments, the clinician can generate the primary assessment and plan based on their observations during the patient visit, while the medical assistant 1120 provides a secondary report, or a second opinion, based on its comparison to the database 1184 of patient embeddings.

In certain embodiments, the assessment and the plan are automatically generated by the analytic module based on the comparison of the patient embedding to the existing patient embeddings where a confidence matching score of the patient embedding to one or more existing patient embeddings is greater than or equal to a second predetermined threshold. In such embodiments, the analytics module 1121 generates only the primary assessment and plan, for example if the confidence score is above a particular threshold (for example, in certain embodiments, higher than 90%), or the clinician agrees with the assessment and plan generated by the medical assistant 1120.

In certain embodiments, the set of post visit administrative instructions can include one or more of inputting or uploading information from the patient post visit record to an electronic heath records database, updating an existing patient record for the patient with information from the patient post visit record, generating a referral letter to a specialty clinician, generating or beginning a pre-authorization process for follow up appointments or procedures, scheduling subsequent appointments for the patient with the clinician or with other clinicians based on information from the patient post visit record, sending a prescription order to a pharmacy based on information from the patient post visit record, coding and/or entering clinician services performed into an electronic billing system, and/or generating and providing patient friendly format of the patient post visit record to the patient before discharge.

In certain embodiments, as discussed further hereinbelow, the post visit administrative instructions can be changed or defined by the information gathered during the patient visit, or may be, at least in part, standardized based on the needs of a particular clinician or particular medical practice. The list may also be, at least in part, informed by historical trends for similar patient embeddings as determined by the analytics module 1121 when comparing to the database 1184.

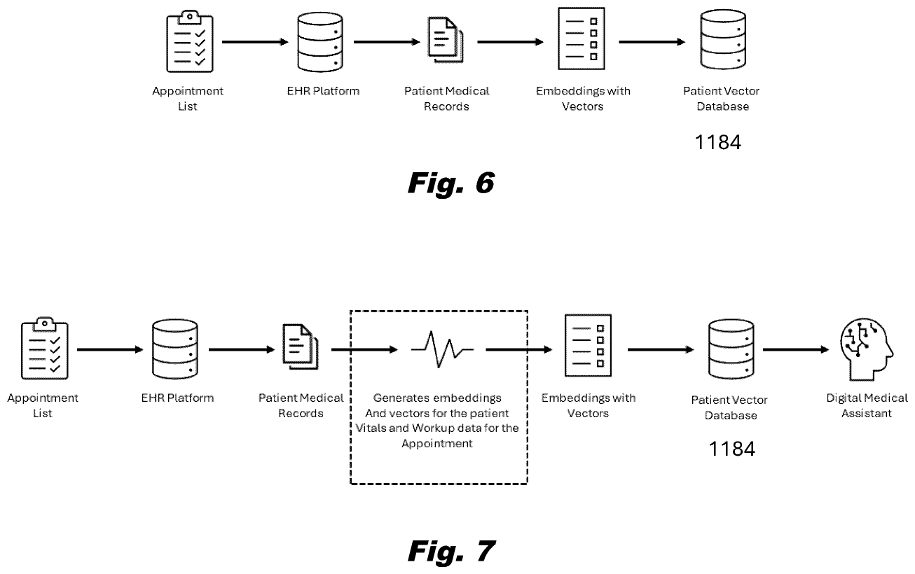

The medical assistant 1120, so far, has been discussed as gathering information (e.g., the input data) in real time during the patient visit. However, in certain embodiments such as shown in FIGS. 6 and 7, the analytic module 1121 is further configured to, in real time, automatically review a patient intake database and scheduling database to determine a list of patients to be seen by the clinician, in other words, perform a pre-appointment service. For each patient in the list, the analytics module 1121 is configured to automatically review an existing patient electronic health record for each patient to be seen, and automatically provide to the clinician, based on the review of the patient intake database and existing electronic health record for a respective patient to be seen, a pre patient visit report for each patient, prior to the respective patient being seen by the clinician.

The pre patient visit report can include one or more of: bibliographic information of the respective patient to be seen, a medical history of the respective patient to be seen; a proposed assessment and plan to be included in the patient post visit record; a list of follow up questions to be asked of the respective patient during the visit; and/or a list of administrative tasks to be completed post patient visit. In certain embodiments, the pre patient visit report can be provided to the clinician in the standardized format, for example, the pre patient visit report can include a draft of the post patient visit report generated based on the intake information for the respective patient.

The pre-visit review can assist the clinician in preparing for the visit and allow the clinician to ask more informed questions of the patient during the review, providing the patient with a more comprehensive and accurate exam. For example, the input module 1130 can generate the patient embedding based on the input data gathered during initial pre-visit review. The analytic module 1121 can then compare the patient embedding to the patient embedding database 1184 before the exam begins to determine a proposed assessment and plan based on the comparison to the patient embedding database 1184, for example using the assessment and plan of patient embeddings with high confidence matching scores.

Once the visit begins, and the patient and clinician begin their dialogue and interaction, as shown in FIG. 7, the analytic module 1121 is further configured to, in real time, automatically modify the assessment and the plan of the pre patient visit report during the patient visit based on the patient embedding and the comparison to the database 1184 of existing patient embeddings if the confidence matching score of the patient embedding relative to one or more existing patient embeddings increases indicating a better fitting assessment and plan compared to the pre patient visit report. The input module 1130 can be continually recognizing the input data in real time during the visit and continually updating or modifying the patient embedding as new data points are recognized, or existing data points change, and the analytics module 1121 can be continually comparing the revised patient embedding to the database 1184 to generate the most accurate post patient visit record in real time. The analytics module 1121 can also update the list of questions for the clinician to ask of the patient as the patient embedding changes during the patient visit. Similarly, the list of administrative tasks to be completed post patient visit can be automatically and continually updated based on the patient embedding and the comparison to the database 1184 of existing patient embeddings.

Turning now to FIGS. 6-9, an exemplary patient visit using the medical assistant 1120 will be discussed. FIG. 6 shows an exemplary process to be performed by the medical assistant 1120 prior to ta patient visit. Before a patient visit, the medical assistant 1120 can, via the input module 1130, automatically review the patient intake system to generate an appointment list for each patient coming to the office for a given day, or other defined time period (e.g., the for the next week or month). The input module 1130 can also review the existing patient health records for each patient included on the appointment list. The patient records can be reviewed directly from a commercially available electronic health records (EHR) platform (e.g., Cerner, Athenahealth, eClinicalWorks, MEDITECH, AdvancedMD, Epic, DrChrono, or the like) or from an internal, locally stored health records database, or both. The input module 1130 reviews the patient records and generates, for each patient, a respective patient embedding. The patient embedding, as described herein, can include a mathematical vector representatively of a plurality of defined datapoints derived from the patient health record. The patient embedding is stored within the patient vector database 1184. The database 1184 can be used during patient visits, as explained next, and can also be used for training purposes, e.g., for training AI models. In certain embodiments, and compliant with electronic health data storage requirements and laws, the patient vector database 1184 can be “global,” in the sense it is not limited to any particular clinician's office, offering a broader database for comparison of patient embeddings, capable of generating more complete and accurate recommendations.

Referring now to FIG. 7, further embeddings can be generated during the visit, or the patient embedding generated pre visit can be updated with information gathered during the visit. For example, the patient embedding can be updated with additional symptoms or complaints provided by the patient that were not included in their intake paperwork. The patient embedding can be updated with lab work, imaging studies, or results of in office tests such as vitals, ultrasounds, or the like. The patient embedding can also be updated with symptoms captured directly by the medical assistant 1120, such as certain acoustics (e.g., wheezing, arrhythmia, percussion sounds, intestinal sounds) and/or certain gestures (e.g., involuntary movements, poor reflexes, lack of movement or range of motion). As shown, the patient embeddings are associated with the respective vectors, and the updated vectors are stored in the database 1184 for use by the medical assistant 1120 in completing later tasks, such as preparing the patient post visit record, or in completing the list of post visit administrative tasks.

FIG. 8 shows an exemplary embodiment of an input device 1110 for gathering input data for use by the input module 1130 and the analytics module 1121. In this example, the input device is a web browser extension 1113. In this example, the clinician has a computer with them during the visit and has the electronic health records platform open to the patient's electronic health record. Here, the browser extension shows that speech recognition is active, and the medical assistant 1120 is capturing the input data, i.e. the dialogue between the patient and the clinician. As shown, the patient has indicated they have a sore throat, and it has been sore for five days. On the display, the electronic health record is automatically updated to include the chief complaint, i.e. sore throat. The subjective assessment field can be pre-loaded, such as with the patient history. In this example, the patients name, age, and sex are pre-loaded from the patient health record. The subjective assessment is automatically updated in real time with new information, i.e., presenting with a sore throat and the patient has had the sore throat for five days. The objective assessment is automatically updated with data collected during the visit, for example vital signs, and tests performed, in this example, checking heart rate and rhythm, and listening to the lungs. The assessment is determined based on the data collected during the exam, in real time. In this example, the clinician has determined the assessment includes a diagnosis of acute pharyngitis, with a plan of bed rest, fluids and follow up. The medical assistant 1120 has concluded, for example based on the information collected and the comparison of said information to the patient embedding database 1184, another cause for these symptoms cause is tonsillitis. This is recorded as a second opinion. Based on the analysis of the patient embeddings in the database 1184, the most likely plan for this secondary assessment is follow up with an ENT specialty clinician.

In this example, the chief complaint, the subjective description, the objective description, the assessment, and the plan are automatically input into the patient's heath record in the EHR platform in real time during the visit. However, it is also possible the medical assistant 1120 can generate the patient post visit record during the visit and store the post patient visit record information until completion of the visit so that the clinician can review and/or approve the report before it is uploaded. At completion of the visit, the medical assistant 1120 can automatically upload the report, or can extract the relevant information from the report, for automatic entry into the patient record in the EHR platform.

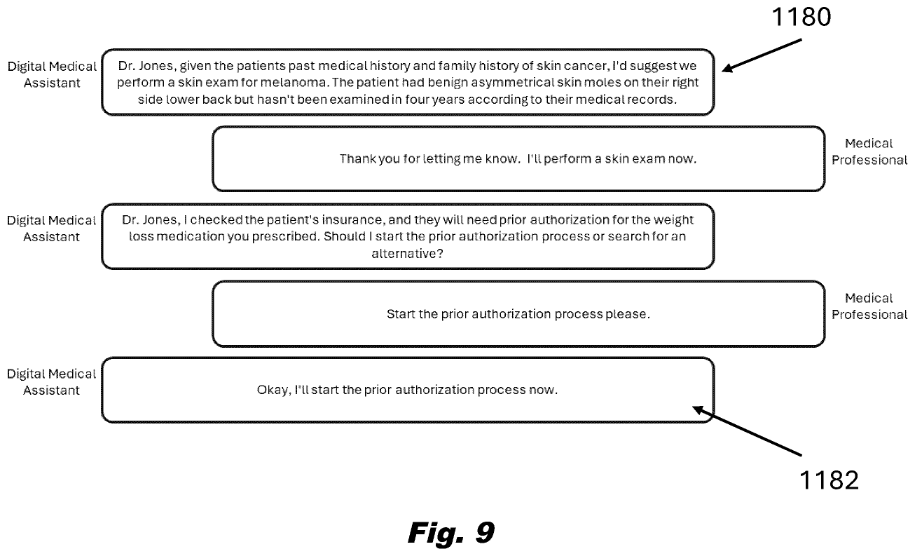

FIG. 9 shows an exemplary embodiment of a two-way interaction between the clinician 1100 and the medical assistant 1120. In this example, the medical assistant 1120 has reviewed the patient's medical history and determined they may be at a higher risk for certain skin cancer. Because of this risk, the medical assistant 1120 suggests to the clinician to perform a skin exam. The dialogue can continue in this fashion as the medical assistant 1120 captures more input data, such as while the patient describes their chief complaint and subjective description of the reason for the visit. The medical assistant 1120 can also inform the clinician in real time whether any obstacles are foreseen for certain procedures. In this example, the medical assistant 1120 has identified the medication to be prescribed requires preauthorization from the insurance company. Here, the clinician can verbally request the medical assistant 1120 begin preparing the preauthorization. In certain embodiments, the medical assistant 1120 can automatically begin preparing the pre-authorization, or similar task, once the obstacle is identified.

With reference now to FIG. 10, an exemplary embodiment of a method 1000 in accordance with at least one aspect of this disclosure, for example a method 1000 for assisting a clinician with a patient visit and associated administrative tasks is shown and described. The method includes, at step 1002 recognizing, in real time, spoken language, i.e. a conversation between a patient and clinician, and converting the spoken language to a computer readable form to generate a patient embedding. The computer readable form can include a mathematical vector associated with the patient embedding. The method further includes, storing the patient embedding in a database 1184 of existing patient embeddings. At step 1004, the method includes employing one or more artificial intelligence based analytical techniques to parse the patient embedding and catalog portions of the patient embedding into a plurality of categories including patient history, patient symptoms, patient condition, patient medication, patient allergy, patient concerns, likely medical billing codes for services rendered during the patient visit. As shown, the one or more AI analytical techniques can include selecting a relevant LLM, for example based on a particular medical specialty relating to the type of patient visit, or a general model.

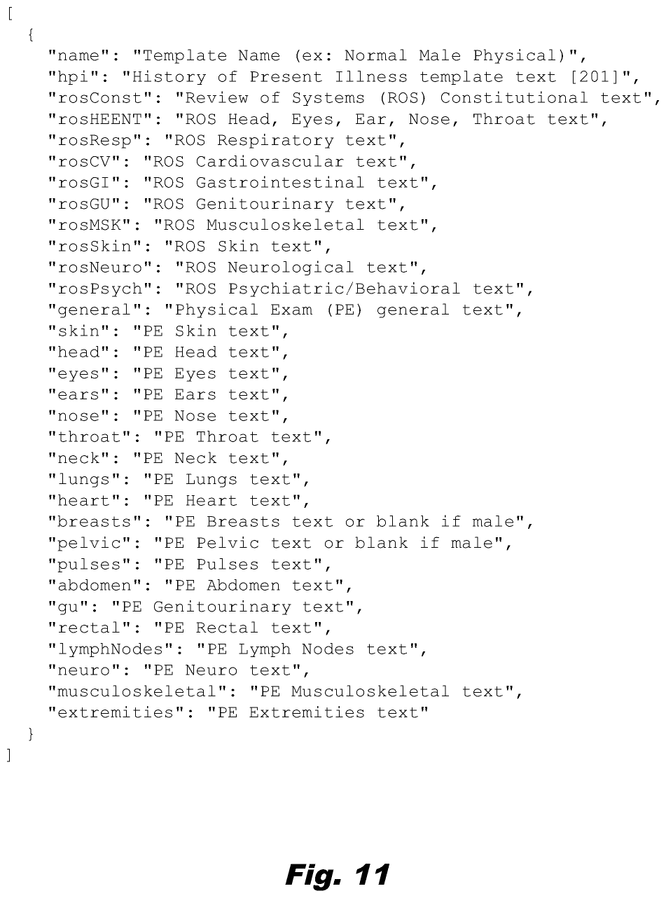

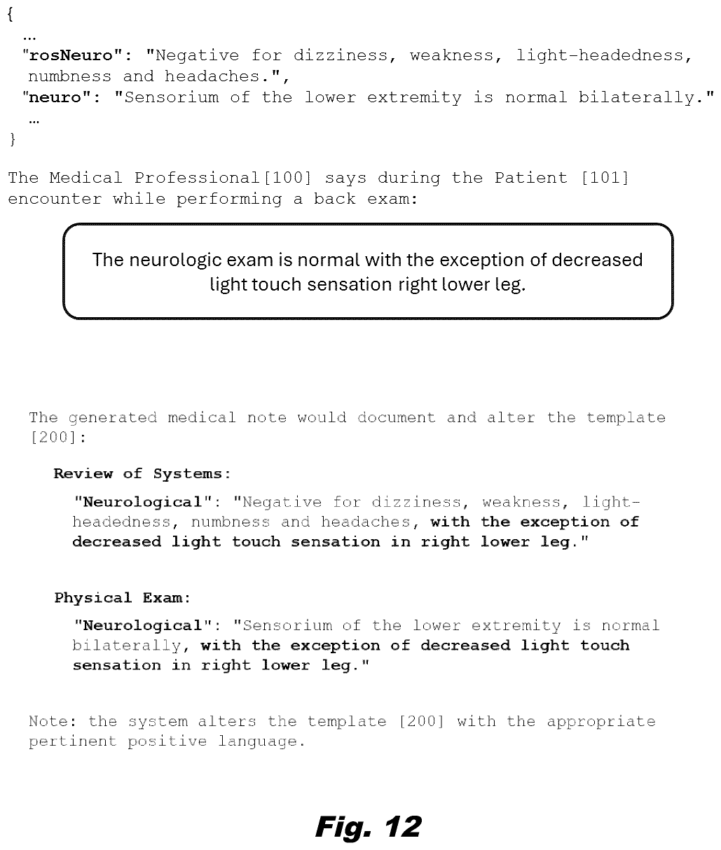

The method includes, at step 1006, comparing the patient embedding to the database 1184 of existing patient embeddings and determining a confidence matching score of the patient embedding relative to one or more existing patient embeddings and automatically generating a set of post visit instructions and automatically generating a patient post visit record based at least in part on one or more existing patient embeddings having a confidence matching score greater than or equal to a first predetermined threshold. The patient post visit record can be presented in a standardized format, e.g., including a subjective description, an objective description, an assessment, and a plan. Steps 1008 to 1018 further describe the generation of the patient post visit record. In certain embodiments, at step 1008, generating the subjective description can optionally include selecting a patient encounter template from a database of stored patient encounter templates. An exemplary embodiment of patient encounter template is shown in FIG. 11. The Patient Encounter Template 1200 can include an array of templates used by the Medical Professional to document different types of Patient encounters. For example, encounters could include normal male or female physicals, normal back exams, normal cough exams, etc. The clinician can use normal templates and the system can document the Pertinent Positives (shown in FIG. 12), which are the differences from the “normal” that the clinician noted during the encounter. For example, a Normal Back Exam medical note could include “The musculoskeletal exam is normal with the exception of positive mild pain on palpation over the right side lower paraspinal muscle” where the medical assistant 1120 would include this observation as part of the automatically generated Musculoskeletal exam medical note from the template. The medical assistant 1120 will recognize the differences from the selected template encounter and automatically update the selected template with the pertinent positives stated during the exam. In certain embodiments, at step 1008, the medical documentation noted during the exam can be stored directly into the EHR platform, without using a template.

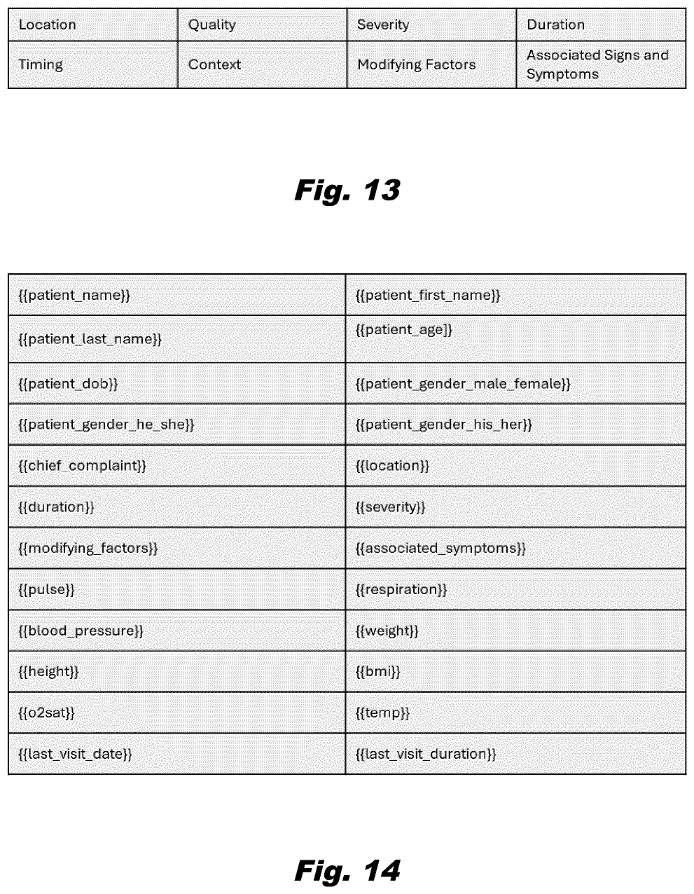

At step 1010, the analytics module 1121 can generate a history of present illness (HPI) based on the patient encounter template selected, and modified in step 1008. The HPI template can allow the Medical Professional to create free-form text and inject elements of the HPI into the HPI narrative. The eight (8) elements of the HPI are shown in FIG. 13. The medical assistant 1120 can also utilize tags to inject data elements into the HPI text. Example tags are shown in FIG. 14. An example of an HPI template for a “Normal Male Physical” is represented below:
“{{patient_first_name}} is a {{patient_age}} {{patient_gender_male_female}} who presents today with complaints of {{chief_complaint}}. {{patient_gender_he_she}} has had this for {{duration}} and rates {{patient_gender_his_her}} pain as {{severity}}. {{history_present_illness}}. Associated symptoms include {{associated_symptoms}}, and modifying factors include {{modifying_factors}}.”

At step 1012, the analytics module 1121 can regenerate the HPI from step 1010 if needed to correct any errors and remove redundancies. At step 1014, the objective description is generated using the patient encounter template described above and shown in FIG. 11. The objective description only includes the pertinent positives noted during the review of symptoms (ROS) as these are the objective findings noted by the clinician during the physical exam (PE). Here, the patient embedding can be updated with the subjective and objective descriptions to provide a better, more accurate assessment and plan based on the comparison of the patient embedding to existing patient embeddings in the database 1184. At step 1016, the analytics module 1121 can perform the comparison and generate the assessment and diagnosis (either as a primary diagnosis, or secondary to the clinicians diagnosis), and further generate the associated billing codes to be included in the list of post visit instructions (i.e. list of administrative tasks).At step 1018 the plan is generated based on the assessment and diagnosis from step 1016, and based on the patient embedding comparison.

At step 1020, the method incudes, providing to the clinician, via one or more different media the patient post visit record in a standardized format, and automatically executing the set of post visit administrative instructions. The output can be provided directly to the clinician in the form of media output such as auditory, visual, or printed materials. Additionally, the output can be directly input into the EHR platform, reducing the administrative burden on the clinician once the patient visit is complete. In certain embodiments, at step 1022, the final patient embedding and associated assessment, plan, and notes generated during the patient visit can be provided to a machine learning service to further train the LLM or other AI models used by the medical assistant 1120 and analytics module 1121. At step 1024, the output module 1140 can provide the patient post visit record to an endpoint within the clinician's organization, such as REST endpoint, webhook, and/or an FTP site, among others as shown in box 1028.

In accordance with at least one aspect of this disclosure, a system can be configured for an AI-driven Mixed-Initiative Dialogue Digital Medical Assistant 1120 in the field of healthcare, allowing medical professionals 1100 to interact with Electronic Health Records (EHRs) 1173, and to perform administrative tasks, utilizing a combination of voice, text, acoustics, and/or gesture for the human-machine interface (HMI). In certain embodiments, the system can be configured to assist medical professionals 1100 in the areas of, but not limited to, medical documentation generation for patient encounters, pharmacy orders, discharge notes, after visit summaries, referral letters, end-of-shift reports, and to schedule follow-up appointments. The system's mixed-initiative dialogue 1152 can be flexible to allow each agent (Medial Professional 1100 or system) to initiate and contribute to the dialogue at any point in time.

The AI-driven Mixed-Initiative Dialogue Digital Medical Assistant 1120 as described herein can revolutionize healthcare by employing advanced artificial intelligence (AI) algorithms to interact with medical professionals 1100 through a combination of voice, text, acoustics, and/or gestures 1103 to assist in performing administrative tasks and appointment workup and review of the patient's past medical history pre-appointment 1180, make recommendations, including a Differential Diagnosis or Second Opinion 1186, during appointment 1181, and post-appointment 1182 tasks. As used herein, appointment means any meeting between a clinician and a patient where testing may or may not be performed. For example, an appointment can include any one or more of the following encounters, a general physical exam (i.e. an annual physical) where no specific tests are performed, a specific physical exam (i.e., a neurological exam, a gastrointestinal exam, or a cardio exam, etc.), a surgery appointment, a testing appointment (e.g., imaging studies, blood work, EEG, ECG, ultrasound, etc.), among others. Certain embodiments of the system can utilize a combination of natural language processing (NLP) 1151, natural language understanding, mixed-initiative conversational dialogue management 1152, speech recognition 131, speech synthesis 1141, image recognition 1133, optical character recognition (OCR) 1133, and large language models 1150 to interpret complex medical terminology, and contextually summarize critical patient information. Certain embodiments can seamlessly integrate with existing EHR systems 1173, automatically populating patient records with accurate and relevant information 1110, thus allowing healthcare providers to focus more on patient care than on administrative tasks.

Certain embodiments of the system can include an input module 1130 and an output module 1140, including Software for Input/Output Devices 1110, which can be or include are a combination of: Mobile Device applications 1111, Web Browser applications 1112, Web Browser Extension applications 1113, Smart Speaker applications 1114, and/or Computer/Medical Equipment applications 1115. These Input/Output Devices can be used to interact with the Medical Professional 1100 and the Patient 1101 to record, playback, upload, and/or display: Speech, Images, Gestures, and/or Acoustic Sounds 1103. For example, speech input for speech-to-text 1131, where the system listens to both the medical professional 1100 and the patient 1101 and transcribes in real-time the conversation utilizing diarization to separate the speakers during the conversation. In certain embodiments, the system (e.g., the input module 1130, can be configured to perform image analysis 1133 including x-ray imagery, echocardiogram, and optical character recognition for reading medical documents. The output module 1140 can be configured to perform image generation output 1142 used to show the medical professional output of image analysis. In certain embodiments, the input module 1130 can be configured to capture Body acoustics 1134 generated from the patient's organs and body systems including lungs, heart, and intestines during an exam, for example during percussion or palpation. In certain embodiments, the input module 1130 can be configured to capture s Gesture recognition 1132 for interacting with the system using a plurality of methods including hand gestures and eye tracking.

The input and output modules 1130, 1140 can be configured to capture the input data, and output data/results, via one or more media devices, including, Mobile devices 1111 for input and output, these may include mobile phones such as Android and iOS phones, smart watch, smart glasses, Mixed Reality, Augmented Reality, Virtual Reality goggles, wearable computers, and tablets such as an iPad or Android tablets; Smart speaker 1114 for Speech input for speech-to-text 131 and for Speech output for text-to-speech 141 from the system; Web browser 1112 with microphone for speech-to-text 1131 and speaker for Speech output text-to-speech 1141 from the system. The Web browser extension 1113 can be configured to listen to the conversation between the Medical Professional 1100 and the Patient 1101, performs real-time Speech-to-Text 1131 transcription, scans the browser HTML DOM of an EHR system to extract patient data, including patent name, date of birth, age, gender and incorporates that patient data when formatting the Subjective (History of Present Illness) response. Additionally, it can scan the browser DOM for the following fields to populate them from the output of the Medical Documentation Generation Service 1170 and Document Service 1143, those fields including, among others, Chief Complaint, Subjective, Objective, Assessment, and Plan (i.e., a standardized “SOAP” documentation). In certain embodiments, the web browser can be configured to integrate with an Electronic Health Record (EHR) webpage. Additional input/output devices can include computer or other medical equipment 1115 for Speech input for speech-to-text 1131 and for Speech output for text-to-speech 1141 from the system and image generation from the system 1142.

Certain embodiments of the medical assistant 1120 can be utilized both during the patient visit, and before a patient visit. Certain embodiments can utilize a Retrieval Augmented Generation (RAG) 1153 methodology for performing pre-appointment services. The Pre-appointment Service allows the Digital Medical Assistant 1120 to automatically create and/or automatically update the patient's embeddings and associated vector database 1184 that will be utilized when prompting the LLM Service 1150 During the Appointment 1181. For example, prior to a patient visit, the medical assistant 1120 can access an appointment list from the EHR 1173 to obtain the patient's reason for the appointment, which may include the patient's Chief Complaint, Annual Exam, etc. The patient's past medical history is converted to vectors and stored in the patient Vector Database 1184. Using a RAG methodology, the input module 1130 can create or update the patient's embeddings and vector database 1184 from the patient's past medical encounters, which can then be used or updated during the patient visit as the clinician and patient converse and as tests are performed.

During the patient visit, the Digital Medical Assistant 1120 can read the patient's workup information, which may include the patient's current vital signs for that appointment, including such data as height, weight, heart rate, respiratory rate, blood pressure, temperature, blood test results, lipid panel results, thyroid test results, blood sugar test results, etc. This step allows the medical assistant 1120 to obtain the most current and relevant information on the patient before the patient speaks with the physician. This information can be analyzed to generate the associated RAG embeddings and vectors 1185 to update the patient Vector Database 1184. The updated Patient Vector Database 1184 is utilized by the Digital Medical Assistant 1120 when generating prompts for the Large Language Model 1150, for example.

During the visit, the medical assistant 1120 can prompt the LLM as discussed here. An exemplary prompt for the LLM using a RAG embedding can be or include,
“Given the patient's past medical history and current vitals, test results, imaging results, which includes {embeddings 1184}, generate a list of potential diagnoses and plans for a {patient_age} {gender}. Format this as a list of JSON objects with the following elements: {“diagnosis_name”, “summary_of_diagnosis”, “clinical_evidence_to_support_your_findings”, “ICD10_billing_codes”, “ICD10_summary”, “confidence_level_as_percentage”, “supporting_medical_citings”, “plan”}”
   In certain embodiments, the patient embedding database 1184 can be local, i.e. only store patient embeddings generated from the clinicians local practice. In certain embodiments, the patient embedding database 1184 can be “global,” for example, when the analytics module 1121 is ready to perform the comparison of the patient embedding to the database 1184, an exemplary follow up prompt to the LLM can be or include:
“Match this patient against national datasets including HealthData.gov, NIH.gov to determine the likelihood of future aliments given the patient's past and current medical data compared against a demographic that is similar to the patient's medical profile, age, and gender.”
   The analytics module 1121 can then evaluate the LLM 1150 responses to generate the Mixed-Initiative Dialogue with the physician 1152. The Digital Medical Assistant 1120 may then initiate a dialogue with the physician to make recommendations on potential health issues that the physician should consider during the appointment, for example by updating the pre-patient visit report or the list of questions generated for the clinician to ask during the visit, based on the pre visit review completed during the pre-appointment service. For example, during the visit, the Digital Medical Assistant 1120 may initiate a dialogue with the physician to inform the physician of anomalies detected in the imaging tests, such as X-rays or MRI results. In certain embodiments, the Digital Medical Assistant 1120 may respond to the physician's requests during the appointment for retrieval of information, such as past medical history, view and analyze imaging data to make recommendations, for example, “Assistant, give me an analysis of the patient's recent imaging results and detect anomalies that I should be aware of.” In certain embodiments, the Digital Medical Assistant 1120 can also initiate a dialogue with the physician to propose a Differential Diagnosis with an associated Plan as a “Second Opinion” for the patient 1186.

Embodiments of the Mixed Initiative Dialogue Service 1152 allows either the Medical Professional 1100, or the system 1120, to initiate a dialogue. The system 1120 can utilize a combination of contextual understand by performing Natural Language Processing 1151 techniques, including Named Entity Recognition (NER), Part of Speech tagging (POS), Large Language Models 1150, and an understanding of the Patient's 1101 past medical, family, social, and surgical history. By reviewing the past medical records, the system assists the Medical Professional 1100 and is able to initiate a dialogue to begin a conversation or respond to a dialogue initiated by the Medical Professional 1100.

The medical assistant 1120 can also assist in the post visit services. For example, as discussed above, the output module 1140 can be configured to automatically execute a set of post visit instructions, which can be or can include a list of administrative tasks. Examples of said administrative tasks can include: generate the medical documentation for the appointment 1170; push the medical documentation to the Electronics Medical Health (EHR) platform 1173; schedule follow-up appointments 1171, which may include medical examinations, testing such as MRI imaging, blood work, surgical appointments, treatment monitoring, and referrals; order pharmacy prescriptions, including refills 1172; check for insurance coverage issues; generate insurance prior authorizations if required; notify physician and office staff of any issues with the post-appointment services that require additional information or processing; and/or notify the patient of post-appointment status, or the like.

Certain embodiments of the system 1120 can include a server architecture that can be hosted in the cloud, on-premises, and/or on-device or a combination thereof. The server architecture can include of a multitude of services that interact and perform tasks in the generation, distribution, and machine learning for medical documentation, pharmacy orders, discharge notes, after visit summaries, referral letters, end-of-shift reports, and scheduling of follow-up appointments.

In certain embodiments, the server architecture 1120 can include a Mixed-Initiative Dialogue Management Service 1152 which can allow either the Medical Professional 1100 or the system to initiate a conversation. The service 1152 can utilize Natural Language Processing 1151, natural language understanding, or similar techniques, to understand the context of the conversation or dialogue. In certain embodiments, the service 1152 can maintain context and may initiate a conversation or dialogue if appropriate. In certain embodiments, the service 1152 can determine that additional information or tasks may need to be performed and may initiate a conversation with the Medical Professional 1100 to obtain additional information or to perform additional task(s).

In certain embodiments, the system can further include a Web Browser Extension 1113 that can interact with the web browser HTML DOM of a 3rd party EHR system, to extract patient information displayed on the web page and to inject the generated medical note 1170 into the appropriate fields on the web page. In certain embodiments, the Web Browser Extension 1113 can allow for the audio recording for Speech-to-Text (Speech Recognition) 1131 of the conversation between the Medical Professional 1100 and the Patient 1101. In certain embodiments, the Web Browser Extension can also allow for Text-to-Speech (Speech Synthesis) 1141 when the system wants to talk to the Medical Professional 1100 and/or the Patient 1101.

To illustrate by way of example, referring back to FIG. 8, the Web Browser Extension 1113 can search for the patient's name, age, gender, and date of birth and utilize that information when generating the medical documentation 1170 and it can listen to the Medical Professional 1100 and the Patient 1101 and perform real-time Speech-to-Text 1131 to obtain a transcription of the encounter. Any one or more additional characteristics can be included in the patient's historical information, including, but not limited to, medical history, medication history, social history, surgical history, and the like. These characterizes can be user defined or can be automatically updated by the analytics module 1121 if it is determined there are relevant characteristics that could be used to provide better diagnoses, or higher confidence matching for patient embeddings. When the medical documentation 1170 (e.g., the patient pre or post visit record) is generated from the speech recognized during the visit (and, in certain embodiments, during the pre-visit review), the Web Browser Extension 1113 can then inject the appropriate medical documentation into the web page(s), which can include, but not limited to, the Chief Compliant, Subjective, Objective, Assessment, and the Plan, e.g., in the same standardized format as the patient post visit record. Additional information can be added to the webpage or

EHR, such as pharmacy orders, lab work orders, lab work results, follow up appointments, tests performed, notes for future insurance authorizations, such as for follow up tests or specialty visits, and the like. For example, the medical documentation 1170 can include, a standard SOAP note, a Progress Note, or any field-level data that can be stored in the EHR (e.g., assessment, ICD-10 billing codes, plan, or the results from a specific test like an eye visual acuity test 20 over 20 (20/20), or intraocular pressure (IOP) of 20 mmHg).

Those having ordinary skill in the art understand that any numerical values disclosed herein can be exact values or can be values within a range. Further, any terms of approximation (e.g., “about”, “approximately”, “around”) used in this disclosure can mean the stated value within a range. For example, in certain embodiments, the range can be within (plus or minus) 20%, or within 10%, or within 5%, or within 2%, or within any other suitable percentage or number as appreciated by those having ordinary skill in the art (e.g., for known tolerance limits or error ranges).

The articles “a”, “an”, and “the” as used herein and in the appended claims are used herein to refer to one or to more than one (i.e., to at least one) of the grammatical object of the article unless the context clearly indicates otherwise. By way of example, “an element” means one element or more than one element.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

Any suitable combination(s) of any disclosed embodiments and/or any suitable portion(s) thereof are contemplated herein as appreciated by those having ordinary skill in the art in view of this disclosure.

The embodiments of the present disclosure, as described above and shown in the drawings, provide for improvement in the art to which they pertain. While the apparatus and methods of the subject disclosure have been shown and described, those skilled in the art will readily appreciate that changes and/or modifications may be made thereto without departing from the scope of the subject disclosure.

What is claimed is:

1. An artificial intelligence driven bi-directional medical assistant, comprising:
an input module configured to recognize, in real time, spoken language and convert the spoken language to a computer readable form to generate a patient embedding, wherein the computer readable form includes a mathematical vector associated with the patient embedding, and wherein the spoken language includes a conversation between a clinician and a patient during a patient visit;
a memory configured to store the patient embedding in a database of existing patient embeddings;
an analytics module configured to, in real time:
use one or more artificial intelligence based analytical techniques to parse the patient embedding and catalog portions of the patient embedding into a plurality of categories including patient history, patient symptoms, patient condition, patient medication, patient allergy, patient concerns, likely medical billing codes for services rendered during the patient visit;
compare the patient embedding to the database of existing patient embeddings and determine a confidence matching score of the patient embedding relative to one or more existing patient embeddings; and
automatically generate a set of post visit instructions and automatically generate a patient post visit record based at least in part on one or more existing patient embeddings having a confidence matching score greater than or equal to a first predetermined threshold; and
an output module configured to, in real time:
provide to the clinician via one or more different media the patient post visit record in a standardized format; and
automatically execute the set of post visit administrative instructions.

2. The assistant of claim 1, wherein the mathematical vector includes a mathematical representation of one or more datapoints in a multidimensional space, the one or more data points including the patient history, the patient symptoms, the patient condition, the patient medication, the patient allergy, the patient concerns, and/or the likely medical billing codes for services rendered during the patient visit.

3. The assistant of claim 1, wherein the input module is configured to convert unstructured data to structured data, wherein the patient embedding is the structured data.

4. The assistant of claim 1, wherein the input module is further configured to, in real time, recognize one or more of:
written language, wherein the written language includes a patient existing record and/or clinician notes generated during the patient visit;
imagery, wherein the imagery includes a patient imaging existing record and/or patient images generated during the patient visit;
gestures, wherein the gestures include gestures performed by a clinician during the patient visit; and/or
non-spoken language acoustics, wherein the non-spoken language acoustics include non-spoken language acoustics generated by the patient during the patient visit.

5. The assistant of claim 4, wherein the input module is configured to perform one or more of: speech recognition, gesture recognition, image recognition, optical character recognition, and/or acoustic recognition on the spoken language, the written language, the imagery, the gestures, and the non-spoken language acoustics, and wherein the input module is configured to convert the written language, the imagery, the gestures, and the non-spoken language acoustics to a respective mathematical vector that is associated with the generated patient embedding, and wherein the analytics module is configured to automatically update the patient embedding with the respective mathematical vectors as the input is captured.

6. The assistant of claim 5, wherein the spoken language, the written language, the gestures, and/or the non-spoken language acoustics are captured by the input module via an input device, wherein the input device includes one or more of a computerized medical equipment, a laptop, a desktop computer, a smart speaker, an internet browser, a mobile device, a tablet, a smart watch, smart glasses, an AR headset, a VR headset, and/or a XR headset.

7. The assistant of claim 4, wherein the output module is configured to perform one or more of: speech synthesis, image generation, and/or document generation to generate and provide the patient post visit record via the one or more different media, wherein the one or more different media include: visual output, haptic output, and/or auditory output.

8. The assistant of claim 7, wherein the output module is configured to provide the visual output and/or auditory output to the clinician via an output device, wherein the output device includes on one or more of a computerized medical equipment, a laptop, a desktop computer, a smart speaker, an internet browser, a mobile device, a tablet, a smart watch, smart glasses, an AR headset, a VR headset, and/or a XR headset.

9. The assistant of claim 1, wherein the analytics modules is further configured to pass the input data to a natural language processing module, a natural language understanding module, a large language model module, a neural network module, a mixed-initiative dialogue manager module.

10. The assistant of claim 9, wherein the standardized format of the patient post visit record includes: a chief complaint, a subjective description, an objective description, an assessment, and a plan, wherein:
the chief complaint, the subjective description, and the objective description are automatically generated from directly the patient embedding prior to the comparison to existing patient embeddings, and
i) the assessment and the plan are automatically generated directly from the patient embedding prior to the comparison to existing patient embeddings, and the analytic module is further configured to automatically generate a secondary patient post visit record including, the chief complaint, the subjective description, the objective description, a secondary assessment, and a secondary plan, wherein the secondary assessment and the secondary plan are automatically generated by the analytic module based on the comparison of the patient embedding to the existing patient embeddings where a confidence matching score of the patient embedding to one or more existing patient embeddings is greater than or equal to a second predetermined threshold, or
ii) the assessment and the plan are automatically generated by the analytic module based on the comparison of the patient embedding to the existing patient embeddings where a confidence matching score of the patient embedding to one or more existing patient embeddings is greater than or equal to a second predetermined threshold.

11. The assistant of claim 1, wherein the set of post visit administrative instructions includes:
input or upload information from the patient post visit record to an electronic heath records database;
update an existing patient record for the patient with information from the patient post visit record;
generate a referral letter to a specialty clinician;
generate or begin a pre-authorization process for follow up appointments or procedures;
schedule subsequent appointments for the patient with the clinician or with other clinicians based on information from the patient post visit record;
send a prescription order to a pharmacy based on information from the patient post visit record;
code and/or enter clinician services performed into an electronic billing system, and/or
generate and provide patient friendly format of the patient post visit record to the patient before discharge.

12. The assistant of claim 1, wherein the analytic module is configured to, in real time,
automatically review a patient intake database and scheduling database to determine a list of patients to be seen by the clinician,
automatically review an existing patient electronic health record for each patient to be seen, and
automatically provide to the clinician, based on the review of the patient intake database and existing electronic health record for a respective patient to be seen, a pre patient visit report for each patient including:
bibliographic information of the respective patient to be seen;
a medical history of the respective patient to be seen;
a proposed assessment and plan to be included in the patient post visit record;
a list of follow up questions to be asked of the respective patient during the visit; and/or
a list of administrative tasks to be completed post patient visit,
wherein the output module is configured to provide the pre patient visit report to the clinician in the standardized format.

13. The assistant of claim 12, wherein the analytic module is configured to, in real time, automatically modify the assessment and the plan of the pre patient visit report during the patient visit based on the patient embedding and the comparison to the database of existing patient embeddings if the confidence matching score of the patient embedding relative to one or more existing patient embeddings increases indicating a better fitting assessment and plan compared to the pre patient visit report.

14. The assistant of claim 12, wherein the analytic module is configured to, in real time, automatically update the list of questions to be asked during the patient visit based on the patient embedding and the comparison to the database of existing patient embeddings.

15. The assistant of claim 12, wherein the analytic module is configured to, in real time, automatically update the list of administrative tasks to be completed post patient visit based on the patient embedding and the comparison to the database of existing patient embeddings, wherein the set of post visit instructions includes the list of administrative tasks to be completed post visit.

16. The assistant of claim 1, wherein the output module is configured to provide the clinician via one or more different media the patient post visit record in a manner that is not readily accessible to the patient during the visit.

17. A method, comprising:
recognizing, in real time, spoken language and converting the spoken language to a computer readable form to generate a patient embedding, wherein the computer readable form includes a mathematical vector associated with the patient embedding, and wherein the spoken language includes a conversation between a clinician and a patient during a patient visit;
storing the patient embedding in a database of existing patient embeddings;
using one or more artificial intelligence based analytical techniques to parse the patient embedding and catalog portions of the patient embedding into a plurality of categories including patient history, patient symptoms, patient condition, patient medication, patient allergy, patient concerns, likely medical billing codes for services rendered during the patient visit;
comparing the patient embedding to the database of existing patient embeddings and determining a confidence matching score of the patient embedding relative to one or more existing patient embeddings;
automatically generating a set of post visit instructions and automatically generating a patient post visit record based at least in part on one or more existing patient embeddings having a confidence matching score greater than or equal to a first predetermined threshold;
providing to the clinician, via one or more different media the patient post visit record in a standardized format; and
automatically executing the set of post visit administrative instructions.

18. The method of claim 17, further comprising, in real time,
recognizing and analyzing written language, wherein the written language includes a patient existing record and/or clinician notes generated during the patient visit;
recognizing and analyzing imagery, wherein the imagery includes a patient imaging existing record and/or patient images generated during the patient visit;
recognizing and analyzing gestures, wherein the gestures include gestures performed by a clinician during the patient visit; and/or
recognizing and analyzing non-spoken language acoustics, wherein the non-spoken language acoustics include non-spoken language acoustics generated by the patient during the patient visit, and
further comprising, converting the written language, the imagery, the gestures, and the non-spoken language acoustics to a respective mathematical vector that is associated with the generated patient embedding; and
automatically updating the patient embedding with the respective mathematical vectors as the written language, the imagery, the gestures, and the non-spoken language acoustics are captured.

19. The method of claim 17, wherein the standardized format of the patient post visit record includes: a chief complaint, a subjective description, an objective description, an assessment, and a plan, and further comprising,
automatically generating the chief complaint, the subjective description, and the objective description directly from the patient embedding prior to the comparison to existing patient embeddings, and
i) automatically generating the assessment and the plan directly from the patient embedding prior to the comparison to existing patient embeddings, and further comprising automatically generating a secondary patient post visit record including, the chief complaint, the subjective description, the objective description, a secondary assessment, and a secondary plan, wherein the secondary assessment and the secondary plan are automatically generated based on the comparison of the patient embedding to the existing patient embeddings where a confidence matching score of the patient embedding to one or more existing patient embeddings is greater than or equal to a second predetermined threshold, or
ii) automatically generating the assessment and the plan based on the comparison of the patient embedding to the existing patient embeddings where a confidence matching score of the patient embedding to one or more existing patient embeddings is greater than or equal to a second predetermined threshold.

20. The method of claim 19, further comprising, in real time,
automatically reviewing a patient intake database and scheduling database and generating a list of patients to be seen by the clinician,
automatically reviewing an existing patient electronic health record for each patient to be seen,
automatically providing to the clinician, in the standardized format, based on the review of the patient intake database and existing electronic health record for a respective patient to be seen, a pre patient visit report for each patient including:
bibliographic information of the respective patient to be seen;
a medical history of the respective patient to be seen;
a proposed assessment and plan to be included in the patient post visit record;
a list of follow up questions to be asked of the respective patient during the visit; and/or
a list of administrative tasks to be completed post patient visit; and
in real time, automatically modifying the assessment and the plan of the pre patient visit report during the patient visit based on the patient embedding and the comparison to the database of existing patient embeddings if the confidence matching score of the patient embedding relative to one or more existing patient embeddings increases indicating a better fitting assessment and plan compared to the pre patient visit report.
